(19) United States
(12) Patent Application Publication
(10) Pub. No.: US 2021/0267257 A1
(43) Pub. Date: Sep. 2, 2021

(54) EXTRUDED PRODUCTS CONTAINING A FIBER PRODUCT

(71) Applicant: Archer Daniels Midland Company, Decatur, IL (US)

(72) Inventors: Michael Fleckenstein, Decatur, IL (US); Michelle Schwenk, Decatur, IL (US)

(73) Assignee: Archer Daniels Midland Company, Decatur, IL (US)

(21) Appl. No.: 17/269,185

(22) PCT Filed: Aug. 19, 2019
(86) PCT No.: PCT/US19/47092

§ 371 Date: Feb. 17, 2021

Related U.S. Application Data
(60) Provisional application No. 62/719,407, filed on Aug. 17, 2018.

(51) Int. Cl. A23P 30/20; A23L 29/225; A23L 29/30; A23L 33/21; A23L 7/17

(52) U.S. Cl. CPC A23P 30/20; A23L 29/225; A23K 10/30; A23L 33/21; A23L 7/17; A23L 29/35

(57) ABSTRACT

In each of its various embodiments, the present invention discloses extruded products containing a dextrin product.

### BACKGROUND OF THE INVENTION

Many raw materials that are used in extrusion to produce extruded products may end up dense, glassy, or have poor expansion if appropriate safeguards are not used. Thinned starches are often used in extrusion to increase expansion and/or modify air cell structure in the extruded matrix of various extruded products. These starches also soften the extruded matrix and decrease the glassy, possibly fractured structure of the matrix.

Fiber is often desired in various extruded products, such as cereals or puffs. However, many of the available fibers may decrease the palatability of the resulting food product. For instance, bran fiber is able to decrease expansion, but often adds a chalky texture to the extruded food product. More specialized fibers such as soluble corn fibers cause decreased expansion and increase the glassy texture of the extruded products. Often, such specialized fibers are also cost prohibitive and need to be combined with other ingredients.

What is desired is an ingredient that is able to function as an extrusion modifier yet is also able to add fiber to an extruded product.

### SUMMARY OF THE INVENTION

In each of its various embodiments, the present invention fulfills this need and discloses extruded food products produced with a fiber containing extrusion modifier, as well as processes for producing such extruded food products.

An extruded product comprising a fiber containing extrusion modifier and a flour is disclosed.

The fiber containing extrusion modifier may have a dextrose equivalent of between about 4 and about 11. The fiber containing extrusion modifier may have at least 50% fiber or even at least 55% fiber. The fiber containing extrusion modifier may comprise digestible starch.

The pH of the fiber containing extrusion modifier may be between about 4 and about 5 in a 20% solution. The fiber containing extrusion modifier may have a viscosity of less than 300 cps in a 30% solution.

The fiber containing extrusion modifier may be present at 5-25% by weight of the extruded product, at 5-15% by weight of the extruded product, or at 7-13% by weight of the extruded product. The flour may be a grain flour or an edible bean flour.

The extruded product may be substantially free of other extrusion aids or texture modifiers, such as calcium carbonate.

A process for producing the extruded product comprises mixing the fiber containing extrusion modifier and the flour, adding water to the fiber containing extrusion modifier and the flour, thus forming a dough, and extruding the dough.

### DETAILED DESCRIPTION OF THE INVENTION

The applicants of the present invention have unexpectedly discovered that by using a fiber containing extrusion modifier, an extruded product may be produced that has an increased level of fiber and increased expansion.

The fiber containing extrusion modifier may be a dextrin having at least about 50% fiber, has a dextrose equivalent of about 4-11, and/or contains starch. The fiber containing extrusion modifier may be produced from the reaction of acid and starch in the presence of heat, thus producing a product that has fiber and starch. The fiber containing extrusion modifier may have a dextrose equivalent of 5-10, a pH in a 20% solution of between about 4 and about 5, a minimum fiber content of at least about 45%, a minimum fiber content of at least about 50%, a minimum fiber content of at least about 55%, a bulk density of about 0.4 g/ml, and/or a viscosity of less than 300 cps in a 30% solution.

The fiber containing extrusion modifier may be used in combination with a flour to produce an extruded product. The use of the fiber containing extrusion modifier enables the production of the extruded product without the use of other extrusion aids or texture modifiers, such as calcium carbonate. Thus, the extruded product produced with the fiber containing extrusion modifier is substantially free of the other extrusion aids or texture modifiers, such as calcium carbonate.

Extruded products containing the fiber containing extrusion modifier may be produced in an extruder.

Also disclosed are foodstuffs into which the extruded products including the fiber containing extrusion modifier have been incorporated.

Also disclosed is an extruded product consisting essentially of a fiber containing extrusion modifier and a flour.

Another embodiment discloses a method of increasing the fiber content of a foodstuff, by incorporating the fiber containing extrusion modifier into the foodstuff. The foodstuff may be a puff, crisp, sheeted cracker, or animal feed.

It should be understood that this invention is not limited to the embodiments disclosed in this summary, or the description that follows, but is intended to cover modifications that are within the spirit and scope of the invention, as defined by the claims.

Other than in the examples described herein, or unless otherwise expressly specified, all of the numerical ranges, amounts, values and percentages, such as those for amounts of materials, elemental contents, times and temperatures of reaction, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (i.e., end points may be used). When percentages by weight are used herein, the numerical values reported are relative to the total weight.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The terms “one,” “a,” or “an” as used herein are intended to include “at least one” or “one or more,” unless otherwise indicated.

Any patent, publication, or other disclosure material, in whole or in part, that is said to be incorporated by reference herein in its entirety is incorporated herein only to the extent that the incorporated material does not conflict with existing definitions, statements, or other disclosure material set forth in this disclosure. As such, and to the extent necessary, the disclosure as explicitly set forth herein supersedes any conflicting material said to be incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The flour used in the extruded products may be a grain flour such as wheat flour (white or whole wheat), corn flour, oat flour, or other type of grain flour. The flour may also be an edible bean flour. The flour may also be a starch, a protein, or other ingredient used in extruded products.

The edible bean from which the edible bean flour originates may be of a Phaseolus species (i.e., beans). In further embodiments, varieties of beans that may be used to produce the bean flours used in the crisps of the present invention include, without limitation, Pinto beans, Great Northern beans, Navy beans, Red beans, Black beans, dark or light Red Kidney beans, Pink beans, MYASI beans, Mayocoba beans, Small Red beans, Black Eyed beans, Cranberry beans, White Beans, Rice beans, Butter beans, African Giraffe beans, and any combinations thereof.

In another embodiment, the edible bean flour may be produced by a milling process to produce the edible bean flour. The edible bean may be subjected to a form of crushing or pulverizing such as by passage of the dehydrated bean through a hammermill or universal mill. In one embodiment, the dehydrated bean product is ground in a PC-20 mill. The ground or powdered dehydrated bean product may be passed through a Swecoscreen 60 mesh, wherein the ground or powdered dehydrated bean product has a particle size of less than about 0.250 mm. In another embodiment, the ground or powdered dehydrated bean product is produced such that about 95% of the ground or powdered dehydrated bean product passes through an 80-mesh screen. The ground or powdered dehydrated bean product may further be passed by a magnet to remove any metallic (i.e., iron-containing) contaminants.

Additional ingredients may be added to the extruded products such as, for example, spices and seasonings, oils, and various processing aids, such as chemical additives which affect functionality and shelf life. Combinations of such ingredients may also be used. Other ingredients that may be added to the foodstuff include, but are not limited to, texture modifiers.

“Seasonings” can include, but are not limited to, minerals such as salt, grain-based seasonings (such as, but not limited to, whole, cracked or ground wheat, corn, oats, rye, flax, barley, spelt and rice), plant-derived seasonings (such as, but not limited to, onion, garlic, pepper, capsicum pepper, herbs, spices, nuts, olives, fruits, vegetables, etc.), and other flavorings (such as, but not limited to, vanilla, sugar, cheese, yeast extract, whey), and combinations thereof.

Vitamins can also be included in the food product such as, but not limited to, niacin, iron, zinc, thiamine mononitrate (vitamin B1), riboflavin (vitamin B2), folic acid, tocopherol(s) (vitamin E), vitamin C, vitamin B6, vitamin B12, vitamin A, vitamin D, pantothenic acid and copper.

The extruded products of the present invention may be made with an extruder. In these types of machines, the dry ingredients may be mixed with water, and the resulting material may be extruded under high temperature and pressure. The material may be extruded out of the machine. When the pressurized material is exposed to atmospheric pressure and ambient temperature, it expands and cools, resulting in a puffed product. The puffed product can be of different shapes and sizes, depending on the die through which it passes and the frequency with which it is cut.

After extrusion, the product may be dried. The final product will have a moisture content of from about 1% to about 8%, depending on the desired characteristics of the finished product.

After production, the extruded products may be packaged for storage and/or sale. Any of the processes described herein may further include at least one of the following acts: placing the extruded products in a container which may be configured for shipping; associating indicia with the container, such as, for example, placing graphical, written, or numerical indicia on the container, wherein the indicia may be capable of describing the contents of the container, designating the producer of the contents, and/or directing an end user, such as, for example, a consumer, on how to use the product; shipping the container containing the product, wherein any conventional method of shipping may be used, such as, for example, shipping by truck, train, ship, or plane; and combinations of any thereof.

The food products produced using the methods described herein can be in the form of crunchy curls, puffs, crisps, protein inclusions, etc. The product can be used as or in a snack food, cereal, or can be used as an ingredient in other foods such as a nutritional bar, breakfast bar, breakfast cereal, or candy.

In various embodiments, the processing conditions and the amounts and types of ingredients can be modified so as to change the nutritional levels of the finished product, as well as for altering the handling, stability, shelf life, texture, flavor, functional properties and ease of manufacture of the product.

The present invention may be further understood by reference to the following examples. The following examples are merely illustrative of the invention and are not intended to be limiting. Unless otherwise indicated, all parts are by weight.

### EXAMPLES

### Example 1

Puffs were produced on a twin-screw extruder with 4 different formulations. The formulations of the puffs were:

1. 100% navy bean flour.

2. 90% navy bean flour and 10% soluble corn fiber.

3. 80% navy bean flour and 20% soluble corn fiber.

4. 95% navy bean flour and 5% FIBERSOL 2 brand maltodextrin.

The twin-screw extruder was a 4-zone extruder with a temperature of about 290° F. at the exit of the extruder. Water was added at a flow rate of about 1 ml/minute. The dry ingredients (flour, fiber, and maltodextrin) were added to the extruder at a flow rate of about 50 lbs./hour.

The soluble corn fiber increased expansion of the puffs by 100% and decreased the bulk density of the puffs by 50% in the finished puffs at the inclusion of 10%. The soluble corn fiber increased expansion of the puffs by 75% and decreased the bulk density of the puffs by 30% at the inclusion of 20%.

### Example 2

In this Example, the following 4 formulations were used to produce puffs.
1. A breakfast cereal formulation including whole oat flour, sugar, salt, calcium carbonate, and a soluble corn fiber.
2. The same formulation as 1, except corn starch was used instead of the soluble corn fiber.
3. The same formulation as 1, except a soluble corn fiber from Tate & Lyle was instead in place of the soluble corn fiber.
4. The same formulation as 1, except a soluble corn fiber from Roquette was instead in place of the soluble corn fiber.

The extruded was a 4-zone extruder having a temperature of about 290° F. at the exit. Water was added at a flow rate of about 1 ml/minutes. The dry ingredients were added to the extruder at a flow rate of about 50 lbs./hour. The bulk density for each of the formulations of this Example was as follows: puff including the corn soluble fiber was about 3.4 lbs./cubic feet; puff including the corn starch has a bulk density of about 2.8 lbs./cubic feet; puff including the Tate & Lyle fiber was about 8.4 lb./cubic feet; and the puff including the Roquette fiber was about 6.2 lbs./cubic feet. The expansion of puff including the soluble corn fiber was more than the starch, and less dense than the fiber from Roquette and Tate & Lyle.

### Example 3

An extruded breakfast cereal formulation including whole corn flour, salt, calcium carbonate, and a soluble corn fiber ingredient was produced. The extruder was a 4-zone extruder having a temperature of about 290° F. at the exit. Water was added at a flow rate of about 1 ml/minutes. The dry ingredients were added to the extruder at a flow rate of about 50 lbs./hour.

### Example 4

Two extruded animal feeds were produced with the same formulation. One animal feed included 10% of the soluble corn fiber ingredient and one animal feed included 10% of chicory root extract. The animal feed including the soluble corn fiber ingredient has a 20% increase in expansion as compared to the animal feed with the chicory root extract and also had less browning.

### Example 5

A sheeted cracker having 10% of the soluble corn fiber ingredient was produced. The sheeted cracker produced with the soluble corn fiber ingredient has a decreased hardness and increased fracturability as compared to a control sheeted cracker produced without the soluble corn fiber ingredient.

While this invention has been particularly shown and described with references to exemplary embodiments thereof, it will be understood by those skilled in the art that various changes in form and details may be made therein without departing from the scope of the invention encompassed by the appended claims.

1. An extruded product comprising:
a fiber containing extrusion modifier; and
flour.

2. The extruded product of claim 1, wherein the fiber containing extrusion modifier has a dextrose equivalent of between about 4 and about 11.

3. The extruded product of claim 1, wherein the fiber containing extrusion modifier has at least 50% fiber.

4. The extruded product of claim 1, wherein the fiber containing extrusion modifier has at least 55% fiber.

5. The extruded product of claim 1, wherein the fiber containing extrusion modifier has a pH of between about 4 and about 5 in a 20% solution.

6. The extruded product of claim 1, wherein the fiber containing extrusion modifier has a viscosity of less than 300 cps in a 30% solution.

7. The extruded product of claim 1, wherein the fiber containing extrusion modifier is present at 5-25% by weight of the extruded product.

8. The extruded product of claim 1, wherein the fiber containing extrusion modifier is present at 5-15% by weight of the extruded product.

9. The extruded product of claim 1, wherein the fiber containing extrusion modifier is present at 7-13% by weight of the extruded product.

10. The extruded product of claim 1, wherein the fiber containing extrusion modifier comprises digestible starch.

11. The extruded product of claim 1, wherein the flour is a grain flour.

12. The extruded product of claim 1, wherein the flour is an edible bean flour.

13. The extruded product of claim 1, wherein the extruded product is substantially free of other extrusion aids or texture modifiers, such as calcium carbonate.

14. A process for producing the extruded product of claim 1, the process comprising:
mixing the fiber containing extrusion modifier and the flour;
adding water to the fiber containing extrusion modifier and the flour, thus forming a dough; and
extruding the dough.
